(19) United States
(12) Patent Application Publication
(10) Pub. No.: US 2023/0060288 A1
(43) Pub. Date: Mar. 2, 2023

(54) MURRAYA KOENIGII EXTRACT AND USE THEREOF IN COSMETICS

(71) Applicant: Gattefossé SAS, Saint-Priest (FR)

(72) Inventors: Virginie CHARTON, Genas (FR); Nicolas BECHETOILLE, Brignais (FR); Frédéric DEMARNE, Marseille (FR)

(73) Assignee: Gattefossé SAS, Saint-Priest (FR)

(21) Appl. No.: 17/797,149

(22) PCT Filed: Jan. 27, 2021
(86) PCT No.: PCT/FR2021/050150

§ 371 Date: Aug. 3, 2022

(30) Foreign Application Priority Data
Feb. 3, 2020 (FR) 2001055

(51) Int. Cl. A61K 8/9789; A61Q 19/08

(52) U.S. Cl. CPC A61K 8/9789; A61Q 19/08; A61K 2800/805; A61K 2800/30

(57) ABSTRACT

The invention relates to the use of a Murraya koenigii extract to combat skin aging.

### FIELD OF THE INVENTION

The invention relates to an extract of Murraya koenigii and a cosmetic composition comprising said extract. The invention also relates to the use of said extract or said cosmetic composition for skin treatment, in particular for skin aging.

### PRIOR ART

The dermis is a dense fibroelastic connective tissue, made up of cells and extracellular matrix (ECM) containing, in particular, elastic and collagen fibers. The function of the dermis is to ensure nutrition and support to the epidermis as well as the mechanical properties of the skin. Fibroblasts, which are the majority cells in the dermis, synthesize the proteins that form the ECM (collagens, elastin, proteoglycans and glycoproteins) but also the proteases (collagenases, elastases) that degrade the ECM in order to renew or repair it.

The genesis of elastic fibers begins in the early stages of development and peaks at birth. This process slows down around puberty and becomes almost non-existent in adulthood. The architecture of mature elastic fibers is complex and tissue-dependent, reflecting the biological function of the tissue under consideration.

In the skin, elastic fibers are present as individualized fibers, giving the skin its elasticity and resilience. Moreover, elastic fibers have different names depending on the proportion of elastin present in these fibers. It is thus possible to differentiate between fibers with a low elastin content and fibers made up of more than 90% elastin; they are respectively called oxytalan fibers, elaunin fibers and mature elastic fibers. In the superficial dermis, oxytalan fibers form fine arborizations perpendicular to the dermal-epidermal junction and intermingle with elaunin fibers in the papillary dermis. In the reticular dermis, the elastic fibers are thicker and oriented parallel to the dermal-epidermal junction.

Elastic fibers are synthesized during elastogenesis, which refers to the set of intra- and extracellular processes leading to the supramolecular assembly of functional elastic fibers. The core of elastic fibers is composed of elastin which is associated with a microfibrillar outer envelope mainly composed of glycoproteins such as fibrillins, fibulins, MAGPs (Microfibril-Associated Glycoprotein-1) and LTBP (LatentTGF-β-Binding Proteins). It is elastin that gives elastic fibers their elasticity and resilience and allows the skin to return to its original position when pinched or stretched.

Elastin is a protein that is very resistant to proteolysis and is among the most resistant proteins in the body. Under physiological conditions in adults, the renewal of elastin is almost nil and its half-life is estimated at 70 years. Nevertheless, elastin synthesis is occasionally reactivated to repair damaged/degraded elastic fibers during aging. However, repair elastic fibers are less functional than those formed during development.

The microfibrils constitute a glycoprotein matrix on which the tropoelastin monomers are deposited and aligned before forming covalent bonds with the maturing fiber. Microfibrils are mainly composed of fibrillins, such as fibrillin-1 (FBN-1) which is highly expressed in the dermis. Fibulins, such as fibulin-5 (FBLN-5), are associated with fibrillins and involved in several key steps of elastogenesis such as binding to tropoelastin and supramolecular assembly of the elastic fiber.

The dermis is affected by chronoaging (also called intrinsic aging) which is the consequence of a set of genetically programmed alterations. Fibroblasts are less numerous, and their metabolic activity is reduced, such as a decrease in collagen and elastin synthesis. Clinically, this results in the appearance of fine wrinkles, a progressive loss of elasticity and slowed healing. During chronoaging, fibroblasts also synthesize more metalloproteinases (MMPs) that are involved in the proteolytic degradation of dermal ECM components. These enzymes are then able to degrade the elastin and fibrillins composing the elastic fibers. From a histological point of view, we examine an elastolysis phenomenon with elastic fibers that appear fragmented with a decrease in their density in number and a decrease in the diameter of these fibers.

Extrinsic aging is the result of many environmental factors, including pollution, lifestyle, and sun exposure (or photoaging), whose harmful effects are added to the effects of chronoaging. Photoaging is characterized by significant alterations in the dermis, which leads to the formation of deep wrinkles and a significant loss of skin elasticity. The changes observed during photoaging are mainly based on the generation of reactive oxygen species (ROS), which will lead to a strong activation of proteolytic enzymes causing excessive tissue degradation. Proteases such as neutrophil elastase (from fibroblasts and neutrophil infiltration) and MMP-12 (from macrophages) are secreted under the influence of UV light to degrade tropoelastin.

With the increase in life expectancy in developed countries, the impact of aging on the appearance and functionality of the skin is a major issue that has been the subject-matter of many studies.

It turns out that a better understanding of the mechanisms involved in the physiology of skin aging allows the development of new strategies capable of slowing or repairing its undesirable effects.

Thus, clinical studies have shown that the application of vitamin C-based creams improves the overall appearance of the skin and reduces marked wrinkles, via the activation of collagen and elastic fiber synthesis.

Retinoids, which are derived from vitamin A, help reduce wrinkles by stimulating the synthesis of collagen fibers and the reformation of oxytalan fibers. In addition, they prevent excessive proteolysis of ECM proteins by blocking the overexpression of MMPs.

Polyphenols, antioxidants naturally present in grapes and wine, green and black teas, fruits, and vegetables, are also good candidates because of their property of inhibiting the overexpression of MMPs.

Plant extracts, which are rich in natural and diverse molecules, are also good candidates for stimulating endogenous mechanisms of photoprotection, such as cellular antioxidant defenses and inhibition of MMPs enzymatic activity. And more particularly, document FR 2855968 describes extracts of dill, currant, cardamom, black radish, little holly, cinnamon, oats, potato, and silk to stimulate the formation of elastic fibers.

The plant species Murraya koenigii (L.) Spreng, commonly known as curry or kaloupile tree for example, belongs to the Rutaceae family. Murraya koenigii or M. koenigii is a deciduous and aromatic shrub that can reach a maximum height of 6 meters. Native mainly to India and Sri Lanka, it is also found in South and Southeast Asia. Its growth is optimal in regions with a tropical or sub-tropical climate.

The phytochemical composition of different parts of M. koenigii, such as the leafy stalk, (or leafy stem), leaf, bark, fruit, seed, root, is well described by the prior art. It is mainly characterized by the presence of alkaloids of carbazole type, phenolic compounds (flavonoids of flavonol type, phenolic acids derived from benzoic acid and p-hydroxycinnamic acid, tannins), terpenoids (saponins, carotenoids), essential oils, minerals, sugars, and amino acids.

M. koenigii, and particularly its leafy stems and leaves, are traditionally used for food and medicine. The leaves are very often used for the preparation of curry and chutney.

Several activities related to cosmetic applications are also reported to M. koenigii in the literature, namely anti-hyaluronidase, antimicrobial, moisturizing, antioxidant, depigmenting, hair growth, or photoprotection.

For example, we can mention the Age Smart® night serum developed by the DERMALOGICA company or the night cream marketed by the ALDI-SUD company. Similarly, reference may be made to US 2009/169651 which discloses a composition comprising plant extracts, including an extract of M. koenigii leaves, and WO 2014/157910 which describes a composition comprising Thanaka obtained by enzymatic treatment of the aerial parts, in particular the leaves, of M. koenigii, M. paniculata, and M. exotica.

In traditional medicine, extracts of Murraya koenigii are said to promote digestion (anti-nausea, anti-vomiting, anti-diarrhea) and combat (myco-)bacterial, fungal, viral, and parasitic infections. Extracts are described as having therapeutic and preventive properties to combat oxidative stress via the stimulation of cellular antioxidant systems. Anti-inflammatory properties are mainly attributed to the alkaloids contained in the Murraya koenigii extracts. Beneficial neurological (anti-anxiety, antidepressant, and anti-stress) and metabolic (anti-diabetic and anti-obesity) effects are also observed in animals fed with Murraya koenigii extracts.

As an example, WO 2018/172436 discloses a composition comprising active ingredients from a dried leaf powder of M. koenigii, including metformin, for use in the treatment of metabolic syndrome and cognitive disorders.

However, it appears from the prior art that the efficacy and biological properties of Murraya koenigii extracts are associated with the presence of alkaloids in said extracts.

Indeed, as mentioned previously, Murraya koenigii is an important source of alkaloids, particularly of the carbazole type. For example, the carbazoles isolated from the stem bark are girinimbine, murrayanine or mahanimbine; the carbazoles mahanimbine, isomahanimbine, mahanine or koenoline are isolated from leaf stems and leaves.

Alkaloids represent a group of molecules of natural origin, containing carbon, hydrogen and, especially, nitrogen. Most alkaloids have a biological action, most often on the nervous system (psychotropic, psychoactive, stimulant, doping, tonic, vomiting, calming, sleeping, and analgesic). In purified form, these molecules very often reveal an acute toxicity, as well as at lower doses, a soothing pharmacological activity, not without habituation effects or a long-term chronic toxicity. Thus, in a cosmetic composition, it is sometimes necessary to reduce the concentration of active ingredients comprising alkaloids in order to reduce the risk of toxicity associated with these molecules. However, this reduction in the dose of active ingredient may be at the expense of the effectiveness of the composition.

From the above, it appears that it is necessary to limit or even eliminate the presence of alkaloids in cosmetic products.

The leaf stalks, (or leafy stems), and leaves also contain phenolic acids derived from benzoic acid (gallic, protocatechuic, syringic acids, . . . ) and p-coumaric acid (p-coumaric, ferulic acids, . . . ) but also flavonoids of the flavonol type (quercetin). These molecules have biological properties useful in the field of cosmetics, in particular, antimicrobial, antioxidant, or even healing properties.

On the other hand, the choice to be made among the available solvents to obtain the extracts poses a certain number of difficulties.

Aqueous solvents require the addition of a microbiological preservative to ensure their stability. However, the field of preservative use is increasingly restricted, particularly because of the toxicological data required. Propylene glycol has been shown to be potentially toxic and is currently being avoided by manufacturers. Agro-based butylene glycol is relatively expensive, which limits its industrial use. Ethanol, although having a good extraction power alone or mixed with water, is particularly volatile and flammable, which leads to transportation and storage problems. On the other hand, because of its drying character, this solvent can disturb the hydrolipidic film of the skin and cause irritations in sensitive skin.

Moreover, as plant extracts are often destined to be used to formulate cosmetic compositions, it is important to avoid extraction solvents comprising salts or acids, of which at least traces will necessarily be found in the extract. Indeed, it is well known that the formulation, in gel or emulsion, of salt-based ingredients beyond a dose of 0.1% is particularly complex. Furthermore, because acids lower the pH of the extracts obtained, they have the effect of making gel or emulsion formulation difficult. In particular, cosmetic products intended for cutaneous application must have a suitable pH, preferably close to that of the skin (about pH 5.5), or neutral. Under these conditions, the use of solvents containing acids therefore requires the addition of excipients, especially pH regulators.

The problem that the invention proposes to solve is that of finding an alternative to existing plant extracts for effectively combating skin aging, in particular by preserving or improving the elasticity of the skin, and which does not have the disadvantages mentioned above, in particular those linked to the presence of alkaloids in the extract.

### DISCLOSURE OF THE INVENTION

The Applicant has found, quite surprisingly, that an extract of Murraya koenigii is able to meet the needs mentioned above.

The invention is therefore directed at the use, preferably non-therapeutic, of an extract of Murraya koenigii according to the invention or of a composition comprising it, for cosmetic treatment of the skin and/or the mucous membranes, in particular for improving the appearance of the skin and/or the mucous membranes, for improving the strength and/or elasticity of the skin, for treating or preventing skin aging, wrinkles, or skin slackening.

According to a first aspect, the invention relates to an extract of Murraya koenigii or a cosmetic composition comprising said extract for combating skin aging.

According to one particular embodiment, the extract according to the invention or the cosmetic composition comprising said extract is used to stimulate the elastogenesis process of the skin.

According to another particular embodiment, the extract according to the invention, the cosmetic composition comprising said extract, is used to stimulate the formation of skin elastic fibers.

Advantageously, the cutaneous elastic fibers are oxytalan fibers, elaunin fibers and/or mature elastic fibers.

According to another particular embodiment, the extract according to the invention, the cosmetic composition comprising said extract, is used to densify the extracellular matrix (ECM) of the skin.

In the sense of the invention, by “densifying the ECM”, we mean increasing the quantity of proteins forming the ECM (collagens, elastin, proteoglycans, or glycoproteins).

According to another particular embodiment, the extract according to the invention or the cosmetic composition comprising said extract is used to combat sagging skin.

According to another particular embodiment, the extract according to the invention or the cosmetic composition comprising said extract is used to heal the skin.

According to one particular embodiment, the Murraya koenigii extract according to the invention implemented in the preceding uses may be obtained by a solid/liquid extraction process, followed by a second solid/liquid separation step and finally by a third recovery step in the liquid phase, characterized in that the solvent consists of a mixture of betaine and 1,3-propanediol or propanediol, advantageously a mixture of betaine, propanediol, and water.

The solvent may or may not contain water. However, the presence of water in the solvent has the advantage of thinning the solvent, and thus facilitating the extraction.

According to one particular embodiment, the extraction solvent used to obtain the extract according to the invention consists of a mixture of betaine and propanediol in molar proportions of 1: strictly less than 1.5; advantageously in molar proportions of 1:1.

According to another particular embodiment, the extraction solvent used to obtain the extract according to the invention consists of a mixture of betaine, propane, and water in molar proportions of 1: strictly less than 1.5 and water represents between 15 and 35% by weight of the solvent.

According to another particular embodiment, the extraction solvent used to obtain the extract according to the invention consists of a mixture of betaine, propanediol, and water in molar proportions of 1:1 and the water advantageously represents between 15 and 35% by weight of the solvent.

Advantageously, the extraction solvent used to obtain the extract according to the invention consists of a mixture of betaine, propanediol and water in molar proportions 1:1:5.

According to a particular embodiment, all or part of the Murraya koenigii plant may be used as an extract to combat skin aging. In practice, the part of the plant used as an extract is selected from the group consisting of the fruit, flower, seed, root, leaf, stem, leafy stalk (or leafy stem), and young shoot.

Advantageously, the part of the plant used is the leafy stalk (or leafy stem), and/or the leaf.

According to a particular embodiment, the plant part used is fresh, frozen, dried, whole, cut, and/or ground.

Advantageously, it is the dried leaf stalk and/or leaf, preferably the dried and ground leaf stalk and/or leaf.

Preferably, it is the dried leaf stalk, preferably the dried and ground leaf stalk.

According to another aspect, the invention relates to a Murraya koenigii extract obtainable by a solid/liquid extraction process, followed by a second solid/liquid separation step and finally by a third liquid phase recovery step, characterized in that the solvent consists of a mixture of betaine and propanediol, advantageously a mixture of betaine, propanediol and water.

According to one particular embodiment, the extraction solvent used to obtain the extract according to the invention consists of a mixture of betaine, propanediol and water in molar proportions 1:1:5.

According to the invention, all or part of the Murraya koenigii plant may be used in the invention. In practice, the part of the plant used in the extraction process is selected from the group consisting of the stalk, leaf, fruit, flower, seed, root, leaf stalk (or leafy stem), and young shoot.

In a particularly preferred manner, the part of the plant used is the leafy stalk and/or the leaf. It may be a fresh, frozen, dry, whole, cut or crushed leaf stem and/or leaf.

Advantageously, it is the dried leaf stalk and/or leaf, preferably the dried and ground leaf stalk and/or leaf.

Preferably, it is the dried leaf stalk, preferably the dried and ground leaf stalk.

According to a particular embodiment, the Murraya koenigii extract according to the invention is free of alkaloids, advantageously carbazoles, preferably mahanimbine, murrayanine, girinimbine, isomahanimbine, mahanine and/or koenoline.

For the purposes of the invention, “alkaloid-free” means an absence of alkaloids in the extract or the presence of trace amounts of alkaloids at a concentration of less than 5 mg/100 g of extract.

According to a particular embodiment, the Murraya koenigii extract according to the invention is rich in phenolic acids, preferably p-hydroxycinnamic acid derivatives, and/or in flavonoids, preferably of the flavonol type.

In the sense of the invention, by “rich” we mean:
a concentration of phenolic acids higher than 100 mg/kg of extract, advantageously higher than 250 mg/kg of extract; and
a flavonoid concentration of more than 50 mg/kg of extract, advantageously more than 100 mg/kg of extract.

The solvent according to the invention is notably liquid at room temperature, which facilitates the implementation of the process leading to the extract of the invention.

In practice, the solvent used may be produced by mixing betaine and propanediol or betaine, propanediol and water in a stirred reactor until a clear, colorless mixture is obtained. This mixing can be done at a temperature between 2° C. and 100° C. and for 30 minutes to 6 hours, preferably 40° C. to 70° C. for 1 hour to 2 hours.

The extract according to the invention is obtainable by a process implementing a solid/liquid extraction step.

The solid/liquid extraction may be performed by different techniques well known to the man of the art, such as maceration, remaceration, digestion, dynamic maceration, decoction, fluid bed extraction, microwave-assisted extraction, ultrasonic-assisted extraction, countercurrent extraction, percolation, re-percolation, leaching, extraction under reduced pressure, diacolation.

In practice, the plant/solvent mass ratio applied for the extraction step is between 1:99 and 50:50, advantageously between 3:97 and 10:90. Preferably, it is a mass ratio of crushed dried Murraya koenigii leaf stalks (or leafy stems) and/or leaves to solvent of between 1:99 and 50:50, preferably between 3:97 and 10:90. The extraction step is preferably carried out at a temperature between 2 and 100° C., more preferably between 20 and 80° C. The extraction step may be maintained for a few minutes to several days.

In order to optimize the extraction of the active compounds while protecting these compounds from oxidation by atmospheric oxygen, the solid/liquid extraction step is advantageously carried out under agitation and/or in a nitrogen atmosphere.

According to the invention, the solid/liquid extraction step is followed by a solid/liquid separation step, the objective being to recover the liquid phase, also called solid/liquid separation filtrate, containing the active material. This separation may be carried out by any technique known to those skilled in the art, in particular draining, pressing, dewatering, centrifuging, or filtration.

Optionally, the liquid/solid separation step may be followed by a concentration step, which allows a concentrate to be obtained, in liquid or semi-solid form depending on the concentration factor. In practice, the concentration step may be performed by evaporation under reduced pressure or by reverse osmosis.

Preferably, the solid/liquid separation filtrate or concentrate additionally undergoes one or more clarification steps. To carry out this clarification step, the skilled person may use any type of frontal and/or tangential filtration known in the field.

According to the invention, the liquid/solid separation or concentration or clarification step may optionally be followed by a fractionation step, which allows a fraction of one or more phytochemical families to be obtained, in liquid or semi-solid form depending on the concentration factor. In practice, the fractionation step may be performed by any technique known to those skilled in the art, in particular by chromatography under reduced pressure, ultrafiltration, or nanofiltration.

Finally, with a view to packaging, the process for obtaining the liquid extract according to the invention may comprise a sterilizing filtration. Sterilizing filtration is traditionally achieved by filtering the product through a filter comprising pores with a diameter of 0.22 μm. Preferably, the sterilizing filtration step is the final step of the process.

The Applicant has also discovered that the Murraya koenigii extract according to the invention has particularly interesting biological properties, especially in terms of stimulating skin protein synthesis.

The Murraya koenigii extract according to the invention is capable of modulating the expression of proteins associated with skin elasticity.

Indeed, as detailed in the experimental section, the Murraya koenigii extract according to the invention has the property of stimulating the expression of proteins belonging to elastic fibers, in particular elastin, fibrillin-1, fibrillin-2 or fibulin-5. The extract according to the invention is therefore particularly interesting for cosmetic uses, in particular for improving the appearance of the skin, or for preserving and/or improving the elasticity of the skin. Consequently, the extract according to the invention makes it possible to combat the effects of skin aging, preferably intrinsic aging, and/or extrinsic aging, in particular, photoaging.

The advantages of the extract according to the invention are:
it has biological (pro-elastogenic) properties in the absence of/with a low alkaloid content;
being totally natural, i.e., all components are natural; and
being compatible and perfectly formulated in all cosmetic care products.

According to another aspect, the invention relates to a composition comprising the extract of the invention, preferably a cosmetic composition, i.e., suitable for topical application to the skin and/or mucous membranes, and/or appendages.

According to one particular embodiment, the extract according to the invention represents between 0.1% and 10% by weight of the composition, advantageously between 0.5% and 5%.

According to one particular embodiment, the composition according to the invention is a skin care composition, an anti-aging skin care composition, or a sunscreen composition.

The composition according to the invention may be in all the galenic forms normally used for topical application to the skin and/or mucous membranes, and/or appendages, for example in anhydrous form, in the form of an oil-in-water emulsion, a water-in-oil emulsion, a multiple emulsion, a silicone emulsion, a microemulsion, a nanoemulsion, a gel, an aqueous solution or a hydroalcoholic solution.

This composition may be more or less fluid and may be, for example, in the form of a cream, an ointment, a milk, a lotion, a serum, or a gel.

The cosmetic composition may contain excipients usually used in the cosmetic fields, such as fats, detergent and/or conditioning surfactants, emulsifiers and co-emulsifiers, hydrophilic or lipophilic gelling agents, preservatives, antioxidants, solvents, exfoliating agents, perfumes, fillers, hydrophilic and lipophilic filters, coloring materials, neutralizers, pro-penetrating agents, and polymers These types of excipients are all well known to the persons skilled in the art.

In practice, the quantities of these different excipients are those conventionally used in the fields considered, and the sum of the excipients preferably represents 0.01% to 30% of the total weight of the composition.

Suitable fats include mineral oils, animal oils (such as lanolin), vegetable oils, synthetic oils (such as isopropyl myristate, octyldodecyl, isostearyl isostearate, decyl oleate, isopropyl palmitate), silicone oils (cyclomethicone, dimethicone) and fluorinated oils. Fatty alcohols, fatty acids, waxes, and gums, and in particular silicone elastomers, may be used as fats.

Suitable detergent and/or conditioning surfactants include nonionic, anionic, cationic, or amphoteric surfactants and mixtures thereof, such as, for example, alkyl sulfates, alkyl ether sulfates such as sodium lauryl ether sulfate, alkyl betaines such as cocamidopropyl betaine, or quaternary ammonium salts.

Suitable emulsifiers and co-emulsifiers include, for example, polyglycerol fatty acid esters, sucrose fatty acid esters, sorbitan fatty acid esters oxyethylenated sorbitan fatty acid esters, PEG fatty alcohol ethers, glycerol fatty acid esters, alkyl sulfates, alkyl ether sulfates, alkyl phosphates, alkyl polyglucosides, dimethicone copolyols.

Suitable hydrophilic gelling agents include, for example, carboxyvinyl polymers (carbomers), acrylic copolymers such as acrylate/alkylacrylate copolymers, polyacrylamides, polysaccharides such as xanthan gum, guar gum, natural gums such as cellulose gum and derivatives, starches and their derivatives, clays, and 2-acrylamido-2-methylpropane acid copolymers.

Examples of suitable lipophilic gelling agents are modified clays such as bentones, metal salts of fatty acids, hydrophobic silica, and ethyl cellulose.

Suitable preservatives include benzoic, sorbic, propionic, salicylic, dehydroacetic acids and their salts, benzyl alcohol, ethylhexylglycerin, parabens, their salts and esters, triclosan, imidazolidinyl urea, 5-phenoxyethanol, DMDM hydantoin, diazolidinyl urea, chlorphenesin.

Examples of suitable antioxidants are chelating agents such as EDTA and its salts, sodium metabisulfite, salicylic, ascorbic, and citric acids and their salts, sodium tartrate, sodium gluconate, carotenoids, and tocopherols.

Solvents that can be used in the cosmetic composition (distinct from the extraction solvent) include water, glycerin, propylene glycol or sorbitol.

Examples of suitable exfoliating agents include chemical exfoliants such as AHAs, and physical exfoliants such as natural or synthetic powders.

Suitable fillers include talc, kaolin, mica, sericite, magnesium carbonate, aluminum silicate, magnesium silicate, organic powders such as nylon.

Suitable dyes include lipophilic dyes, hydrophilic dyes, pigments, and pearlescent materials commonly used in cosmetic compositions and mixtures thereof.

Suitable neutralizers include soda ash, triethanolamine, aminomethyl propanol or potassium hydroxide.

Suitable pro-penetrating agents are, for example, alcohols and glycols (ethanol, propylene glycol), ethoxydiglycol, alcohols and fatty acids (oleic acid), fatty acid esters or dimethyl isosorbide.

The composition of the invention may also contain active ingredients other than the extract according to the invention. As appropriate active ingredients, we can mention for example the anti-radicals or more generally the antioxidants, whitening agents, pigmenting agents, emollients, moisturizers, anti-seborrheic agents, anti-inflammatories, anti-acne agents, keratolytic and/or desquamating agents, anti-wrinkle and tensor agents, draining agents, anti-irritant agents, soothing agents, vitamins and their mixtures, mattifying agents, anti-aging agents such as retinol, healing agents, antiseptics, and essential oils.

The invention and the advantages thereof will become apparent from the following embodiments.

### BRIEF DESCRIPTION OF THE FIGURES

The manner in which the invention may be carried out and the advantages thereof will become clearer from the following illustrative and non-limiting examples, which are shown in the attached figures.

FIG. 1 represents the content of phenolic acids, flavonoids, and alkaloids of 4 extracts of Murraya koenigii.

### DETAILED DESCRIPTION OF THE INVENTION

1/ Preparation of Murraya koenigii Extracts

Extract 1: Extract of Murraya koenigii obtained from dried and ground leaf stems and leaves by extraction with a solvent according to the invention consisting of a mixture of betaine, propanediol and water in a molar ratio of 1:1:5 (GHP115) under continuous stirring for 3 hrs. at 70° C. of the plant and the solvent in the mass ratio 8:92. After extraction, the plant residue is removed by a mechanical process (e.g., spinning) and the crude extract is filtered down to 0.22 μm.

Extract 1a: Extract obtained under the same operating conditions as described for Extract 1 above, except that the plant/solvent mass ratio is 5:95.

Extract 2: Extract of Murraya koenigii obtained from dried and ground leaf stalks and leaves by extraction with an ethanol/water solvent in a 70:30 mass ratio under continuous stirring for 3 hrs. at 70° C. of the plant and the solvent in the 8:92 mass ratio. After extraction, the plant residue is removed by a mechanical process (e.g., spinning). The crude extract is decolorized on activated carbon Acticarbone CPW CECA at 0.08% then the ethanol is evaporated under reduced pressure and the aqueous concentrated extract obtained is solubilized in propanediol such that the final dry extract is equal to the dry extract of the hydro-ethanolic extract.

Extract 3: Fraction of the decolorized hydroethanol extract described for Extract 2 enriched in polyphenols and containing only traces of alkaloids (<10 mg/100 g fraction). The fraction is obtained by column filtration on Amberlite FPX 68 resin of the 70:30 decolorized ethanol/water extract on activated carbon, as described for Extract 2, the non-adsorbed filtrate constitutes Extract 3.

Extract 4: Fraction of the decolorized hydroethanol extract described for Extract 2 enriched in alkaloids and devoid of polyphenols. The fraction is obtained by filtration/adsorption on Amberlite FPX 68 resin in column of the 70:30 ethanol/water extract and decolorized on activated carbon as described for Extract 2, then desorption with ethanol of molecules adsorbed on the resin.

2/ Characterization of the Extraction Power of Murraya koenigii by the Solvent Betaine/Propanediol/Water in Different Molar Proportions

The extractability of polyphenols and alkaloids from leaf stalks (or leafy stems) and leaves of Murraya koenigii was studied for 6 compositions of betaine/propanediol/water mixtures (in different molar ratios), shown in Table 1.

TABLE 1

| GHP Molar Ratio | 1:1:4 | 1:1:5 | 1:1.5:3 | 1:1.5:5 | 1:2:3 | 1:2:6 |
|---|---|---|---|---|---|---|
| Betaine (g) | 44.16 | 41.35 | 41.06 | 36.45 | 36.23 | 31.04 |
| Propanediol (g) | 28.68 | 26.86 | 40.00 | 35.52 | 47.06 | 40.32 |
| Water (g) | 27.16 | 31.79 | 18.94 | 28.03 | 16.71 | 28.64 |

Each solvent is prepared by mixing betaine, propanediol and water at 70° C. for about 1 hr under mechanical stirring. Each extract is obtained by extraction at 70° C. for 4 hrs. of 5% of crushed Murraya koenigii leaf stalks and dry leaves and 95% of solvent. After extraction, the plant residue is removed by a mechanical process and the crude extract is filtered to the sterilizing filtration limit (0.22 μm).

Flavonoids and phenolic acids (belonging to the polyphenol group) were analyzed by RP-UHPLC-UV, phenolic acids were spotted and quantified against the calibration line of chlorogenic acid at 287 nm and flavonoids were identified and quantified against the isoquercitrin calibration line at 355 nm. The Gardner index of each extract was measured after dilution to 1/2 m/v in water with a Lico 100 colorimeter.

The physical stability (visual aspect) of the 6 extracts packaged in glass bottles was observed after 7 days of storage at 3 different temperatures (4, 25 and 40° C.).

The flavonoid and phenolic acid concentrations for each extract are represented in Table 2.

TABLE 2

| | Concentration (mg/g extract) | | |
|---|---|---|---|
| | | Phenolic | Gardener |
| Solvent | Flavonoids | Acids | Index |
| GHP 1:1:4 | 11 | 24 | 10 |
| GHP 1:1:5 | 12 | 27 | 9 |
| GHP 1:1.5:3 | 10 | 21 | 8 |
| GHP 1:1.5:5 | 11 | 24 | 10 |
| GHP 1:2:3 | 11 | 20 | 7 |
| GHP 1:2:6 | 12 | 25 | 11 |

The 6 solvents show similar flavonoid extraction performance, characterized by a concentration between 10 and 12 mg/100 g of extract. As for their extraction performance with respect to phenolic acids, it is between 20 and 27 mg/100 g of extract.

All of the solvents tested in this example are effective in obtaining an extract of Murraya koenigii comprising an effective number of flavonoids and phenolic acids for use in cosmetics. The most efficient solvent for the extraction of flavonoids and phenolic acids is the solvent consisting of a mixture of betaine, propanediol and water in molar proportions 1:1:5.

3/ Comparison of the Phenolic Acid, Flavonoid and Alkaloid Content of the Murraya koenigii Extract According to the Invention with Other Extracts

The alkaloid, phenolic acid, and flavonoid contents of the 4 Murraya koenigii extracts described in Example 1 were evaluated by RP-HPLC-UV. Phenolic acids were identified and quantified against the neochlorogenic acid calibration line at 287 nm, flavonoids were spotted and quantified against the isoquercitrin calibration line at 355 nm, and alkaloids were identified and quantified against the mahanimbine calibration line at 287 nm.

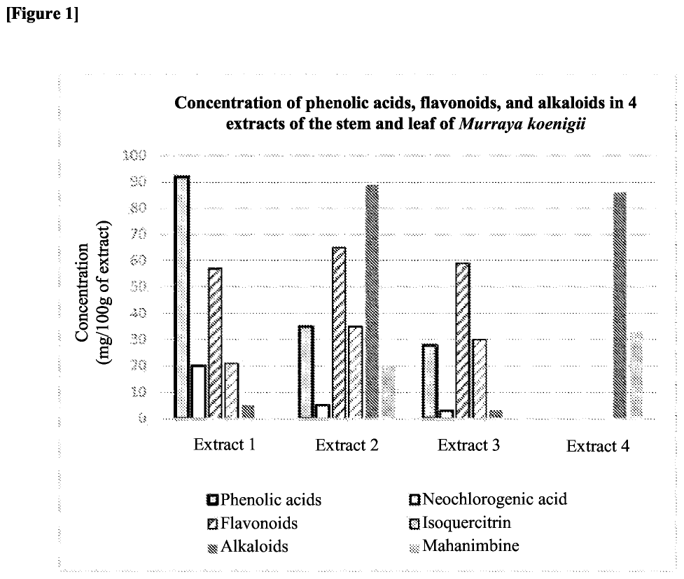

The results obtained are illustrated in FIG. 1.

The composition of the 4 extracts is clearly distinct from a qualitative and quantitative point of view concerning the 3 families of secondary metabolites studied. Thus:
Extract 1 (GHP115 according to the invention) is characterized by the highest concentration of phenolic acids, a concentration of flavonoids equivalent to that of extracts 2 and 3, and the presence of traces of alkaloids;
Extract 2 is characterized by a significant concentration for each of the 3 families of compounds (phenolic acids, flavonoids, and alkaloids);
Extract 3 is characterized by a similar concentration of polyphenols as extract 2, but contains only traces of alkaloids; and
Extract 4 is characterized by a similar concentration of alkaloids as extract 2, but does not contain polyphenols.

From the foregoing, it is clear that only the solvent according to the invention consisting of a mixture of betaine, propanediol and water in molar proportions of 1:1:5 makes it possible to obtain an extract of Murraya koenigii rich in flavonoids and phenolic acids and having a very low content of alkaloids, in the trace state.

4/ Comparison of Phenolic Acid, Flavonoid and Alkaloid Content in the Murraya koenigii Extract According to the Invention with the Plant/Solvent Ratio

The alkaloid, phenolic acid and flavonoid contents of extracts 1 and 1a of Murraya koenigii described in Example 1 were evaluated by RP-HPLC-UV under the conditions described in Example 3.

The results are described in Table 3 below.

TABLE 3

| | | Concentration (mg/100 g of the Extract) | | |
|---|---|---|---|---|
| | Plant/Solvent | Phenolic | | |
| Extract | Ratio | Acids | Flavonoids | Alkaloids |
| Extract 1a | 5:95 | 45 | 26 | Traces |
| Extract 1 | 8:92 | 92 | 57 | 5 |

It appears from the above that the reduction of the plant/solvent ratio to 5:95 (Extract 1a), compared to the 8:92 ratio (Extract 1), makes it possible to obtain an extract containing only unquantifiable traces of alkaloids while presenting an interesting concentration of phenolic acids and flavonoids.

5/ Study of the Effect of Murraya koenigii Extract According to the Invention (Extract 1) on the Expression of Proteins Associated with the Formation of Elastic Fibres

5.1/ Method

The protein content (proteome) and its expression variations in normal human dermal fibroblasts treated or not with the 4 extracts of Murraya koenigii described in Example 1 (extracts 1, 2, 3 and 4) were identified by the nano-liquid chromatography-tandem mass spectrometry (nano LC-MS/MS) method. This analytical method is quantitative, sensitive, and reproducible and allows the identification of proteins expressed in cells by a bioinformatics analysis (CORAVALID).

5.2/ Protocol

The proteomic study consists of qualitative and quantitative analysis of proteins expressed in untreated (UT) cells compared to cells treated with the 4 Murraya koenigii leaf extracts described in Example 1. The 4 extracts are tested at their maximum non-cytotoxic concentration, i.e., Extract 1 (invention) at 0.2%; Extract 2 (propanediol/water) at 0.04%; Extract 3 (ethanol/water) at 1% and Extract 4 (ethanol) at 0.1%, and incubated in the cell culture medium for 24 hrs.

The fibroblasts are then washed with cold phosphate-saline buffer, recovered and frozen at −80° C. Protein lysates and corresponding peptide extracts are obtained according to the method described by Wisniewski J R et al, Nat Methods. 2009; 6(5):359-62.

250 ng of peptides are injected into a C18 pre-column (300 μm×5 mm; particle diameter 5 μm; Thermo Scientific) and then eluted into a C18 column (75 μm×500 mm; particle diameter 2 μm; Thermo Scientific). Liquid nano-chromatography is performed with the Ultimate 3000 equipment (Dionex) by applying a 60-35% acetonitrile gradient for 60 minutes at a flow rate of 300 nl/min. Data are generated with the Q-Exactive mass spectrometer (Thermo Scientific).

The proteome of the cells is analyzed using the SEQUEST-HT algorithm on the UNIPROT database. Protein quantification is performed by the Minora algorithm and the ratio of cells treated with Murraya koenigii extract/untreated cells is calculated using the Pairwise Ratio Based method and then expressed as the geometric median of all generated data.

The study is based on 3 independent experiments (n=3). The hypothesis test performed was an analysis of variance (ANOVA) to compare the proteome of untreated fibroblasts versus fibroblasts treated with the 4 Murraya koenigii extracts described in Example 1.

5.3/ Results

The study identified 4 proteins associated with elastic fibers and whose expression was significantly increased in dermal fibroblasts treated with the betaine/propanediol/water extract (molar ratio 1:1:5) of Murraya koenigii.

The results are presented in Table 4 and are expressed as the treated cell expression ratio with a Murraya koenigii extract/untreated cells and averaged over the 3 experiments.

TABLE 4

| | Ratio Extract | |
|---|---|---|
| Protein | 1/Untreated | p Value |
| Elastin | 2.18 | 6.6E−14 |
| Fibrillin-1 | 2.18 | 1.0E−17 |
| Fibrillin-2 | 2.07 | 2.8E−12 |
| Fibulin-5 | 1.69 | 9.4E−11 |

Surprisingly, only the Murraya koenigii extract according to the invention (Extract 1: GHP115) significantly increases the protein expression of elastin and its 3 partners, fibrillin-1, fibrillin-2, and fibulin-5 in normal human dermal fibroblasts. The other 3 extracts (Extract 2: propanediol/water; Extract 3: ethanol/water and Extract 4: ethanol) have no effect on elastic fiber proteins (data not shown).

5.4/ Conclusion

The extract of Murraya koenigii according to the invention, i.e., obtained by means of the solvent consisting of a betaine/propanediol/water mixture (molar ratio 1:1:5; Extract 1) increases the expression of proteins belonging to the elastic fibers. Normal human dermal fibroblasts treated with the Murraya koenigii betaine/propanediol/water extract according to the invention thus exhibit a phenotype favorable for the regeneration and renewal of skin elastic fibers.

6/ Study of the Effect of Murraya koenigii Extracts (Extract 1 and La) on the Synthesis of Elastin, Fibrillin-1, and Fibulin-5 Synthesis in 2D Culture of Normal Human Fibroblasts.

6.1/Method

This study uses in situ fluorescent immunostaining coupled with image analysis to measure elastin, fibrillin-1 and fibulin-5 syntheses deposited in monolayer cultures of dermal fibroblasts treated or not with the Extracts 1 and 1a according to the invention (betaine/propanediol/water, molar ratio 1:1:5) of Murraya koenigii.

6.2/ Protocol

Normal human dermal fibroblasts are treated for 48 hrs. with the extracts according to the invention (betaine/propanediol/water and molar ratio 1:1:5) of Murraya koenigii obtained in Example 1 (Extracts 1 and 1a), at the final concentration of 0.2%. Untreated (UT) cells are used as controls. The cells are then washed with phosphate-saline buffer before being fixed and saturated with 1% BSA (bovine serum albumin) for 30 minutes. Cells are incubated with the primary antibody (anti-elastin; anti-fibrillin-1 or anti-fibulin-5) for 1 hour, washed in PBS buffer, and then incubated with the Alexa 594 fluorochrome-bound secondary antibody for 1 hour. The fluorescence is read on a Cytation 5 imaging spectrophotometer (Biotek) with the appropriate filters. Fluorescence measurements by image analysis are performed on common acquisition parameters.

The study is based on 3 independent experiments (n=3). The statistical test is the 2 Way-ANOVA followed by Bonferroni multiple comparisons to compare the synthesis of proteins of interest deposited in untreated dermal fibroblast cultures versus fibroblasts treated with Extracts 1 and 1a (betaine/propanediol/water, molar ratio 1:1:5) of Murraya koenigii according to the invention.

6.3/ Results

6.3.1/ Elastin Synthesis

We quantified elastin synthesis deposited in dermal fibroblasts treated or not with Extracts 1 and 1a (betaine/propanediol/water, molar ratio 1:1:5) of Murraya koenigii. The results are presented in Table 5 and expressed as a % of the untreated cells (UT). Values are expressed as ±the mean standard deviation in all 3 experiments (n=3).

TABLE 5

| | Untreated | Extract 1 | Extract 1a |
|---|---|---|---|
| Exp_1 | 100 ± 34 | 158 ± 38 | 150 ± 22 |
| Exp_2 | 100 ± 21 | 151 ± 26 | 168 ± 27 |
| Exp_3 | 100 ± 23 | 178 ± 21 | 172 ± 20 |

| | | |
|---|---|---|
| n = 3 | 62% ± 14 | 63% ± 12 |
| Statistics: ****: p < 0.0001 | **** | **** |

In an equivalent manner, the Murraya koenigii Extracts 1 and 1a obtained according to the invention (betaine/propanediol/water, molar ratio 1:1:5) induce a significant increase in the protein synthesis of deposited elastin (+62% and +63%, respectively) within the dermal fibroblasts.

The extraction solvent alone (betaine/propanediol/water 1:1:5 molar ratio without Murraya koenigii) does not induce an increase in deposited elastin synthesis (data not shown).

6.3.2/ Synthesis of Fibrillin-1

We quantified the synthesis of fibrillin-1 deposited in dermal fibroblasts that were treated or not with Murraya koenigii Extracts 1 and 1a (betaine/propanediol/water, molar ratio 1:1:5). The results are presented in Table 6 and expressed as a % of the untreated cells (UT). Values are expressed as ±the mean standard deviation in all 3 experiments (n=3).

TABLE 6

| | Untreated | Extract 1 | Extract 1a |
|---|---|---|---|
| Exp_1 | 100 ± 11 | 189 ± 16 | 152 ± 30 |
| Exp_2 | 100 ± 32 | 129 ± 44 | 137 ± 26 |
| Exp_3 | 100 ± 43 | 140 ± 38 | 128 ± 33 |

| | | |
|---|---|---|
| n = 3 | 53% ± 32 | 39% ± 12 |
| Statistics: ****: p < 0.0001 | **** | ** |

The Murraya koenigii Extracts 1 and 1a obtained according to the invention (betaine/propanediol/water, molar ratio 1:1:5) induce a significant increase in protein synthesis of fibrillin-1 deposited in dermal fibroblasts. Extracts 1 and 1a are not statistically different from each other even if Extract 1 increases (+53%) the protein synthesis of fibrillin-1 in dermal fibroblasts more strongly than Extract 1a (+39%).

An extraction solvent alone (betaine/propanediol/water 1:1:5 molar ratio without Murraya koenigii) does not induce an increase in deposited fibrillin-1 synthesis (data not shown).

6.3.3/ Synthesis of Fibrillin-5

We quantified the synthesis of fibulin-5 deposited in dermal fibroblasts treated or not with Extracts 1 and 1a (betaine/propanediol/water, molar ratio 1:1:5) of Murraya koenigii. The results are presented in Table 7 and expressed as a % of the untreated cells (UT). Values are expressed as ±the mean standard deviation in all 3 experiments (n=3).

TABLE 7

| | Untreated | Extract 1 | Extract 1a |
|---|---|---|---|
| Exp_1 | 100 ± 53 | 260 ± 121 | 162 ± 56 |
| Exp_2 | 100 ± 56 | 222 ± 133 | 196 ± 74 |
| Exp_3 | 100 ± 36 | / | 221 ± 131 |

| | | |
|---|---|---|
| n = 3 | 141% ± 27 | 93% ± 30 |
| Statistics: ****: p < 0.0001 | **** | **** |

The Murraya koenigii Extracts 1 and 1a obtained according to the invention (betaine/propanediol/water, molar ratio 1:1:5) induce a significant increase in protein synthesis of fibrillin-5 deposited in dermal fibroblasts. Extract 1 increases (+141%) the protein synthesis of fibulin-5 in dermal fibroblasts more strongly than extract 1a (+93%).

The extraction solvent alone (betaine/propanediol/water 1:1:5 molar ratio without Murraya koenigii) does not induce an increase in Fibulin-5 synthesis (data not shown).

6.4/ Conclusion

Extracts 1 and 1a (betaine/propanediol/water, molar ratio 1:1:5) of Murraya koenigii according to the invention induce an improvement in the protein synthesis of the elastic network in the dermis.

7/ Example of Composition—Natural Moisturizing Cream Gel

TABLE 8

| | Composition |
|---|---|
| Name of Ingredients | (% by mass) |
| Emulium ® Mellifera | 2.5 |
| Natural squalane | 2.0 |
| Dicaprylyl carbonate | 15.0 |
| Demineralized water | QS100 |
| Glycerin | 20.0 |
| Tapioca starch | 5.0 |
| Murraya koenigii extract | 2.0 |
| according to the invention | |
| Ethylhexylglycerin | 0.1 |
| Phenoxyethanol | 0.9 |
| Perfume | 0.2 |
| Total | 100 |

1. A method of combatting skin aging, the method comprising topical application to the skin and/or mucous membranes, and/or appendages an extract of Murraya koenigii obtainable by a solid/liquid extraction process, followed by a second solid/liquid separation step and finally by a third step to recover the liquid phase, characterized in that the solvent consists of a mixture of betaine, propanediol and water, in which the betaine:propanediol molar proportions are 1: strictly less than 1.5; or of a composition comprising said extract.

2. A method according to claim 1, for wherein combatting skin aging comprises stimulating the process of skin elastogenesis.

3. A method according to claim 1, wherein combatting skin aging comprises for stimulating the formation of skin elastic fibers.

4. A method according to claim 1, wherein combatting skin aging comprises densifying the cutaneous extracellular matrix.

5. A method according to claim 1, wherein combatting skin aging comprises combatting skin slackening.

6. A method according to claim 1, wherein the solvent consists of a mixture of betaine, propanediol and water, wherein the molar proportions of betaine:propanediol are 1:1.

7. A method according to claim 1, wherein the water represents between 15 and 35% by weight of the solvent.

8. A method according to claim 6, wherein the solvent consists of a mixture of betaine, propanediol and water in molar proportions 1:1:5.

9. An extract of Murraya koenigii obtainable by a solid/liquid extraction process, followed by a second solid/liquid separation step and finally by a third liquid phase recovery step, characterized in that the solvent consists of a mixture of betaine, propanediol, and water, advantageously in molar proportions of 1:1:5.

10. An extract according to claim 9, being an extract of leaf stalks and/or leaves advantageously dried.

11. An extract according to claim 9, being free of alkaloids.

12. An extract according to claim 9, having a mass ratio of leaf stems and/or leaves of Murraya koenigii/solvent of between 1:99 and 50:50.

13. A cosmetic composition comprising the extract according to claim 9.
